(19) United States
(12) Patent Application Publication
(10) Pub. No.: US 2018/0163167 A1
(43) Pub. Date: Jun. 14, 2018

(54) METHOD FOR PREPARING INTRACELLULAR ENZYMES

(71) Applicants: Amano Enzyme Inc., Nagoya-shi (JP); National University Corporation Yamagata University, Yamagata-shi (JP)

(72) Inventors: Yasushi Minamitani, Yonezawa-shi (JP); Toshiyuki Sugiura, Kitanagoya-shi (JP)

(21) Appl. No.: 15/577,815

(22) PCT Filed: May 26, 2016
(86) PCT No.: PCT/JP2016/065639

§ 371 Date: Nov. 29, 2017

(30) Foreign Application Priority Data
May 29, 2015 (JP) 2015-110689

(51) Int. Cl. C12N 1/06; C12N 13/00; C12N 1/16

(52) U.S. Cl. CPC C12N 1/063; C12N 13/00; C12M 35/02; C12N 1/16; C12Y 302/01108

(57) ABSTRACT

The present invention addresses the problem of preparing an intracellular enzyme of yeast by a simple method. A pulsed electric field is applied to yeast, and the enzyme extracted into an extracellular solution is recovered.

### TECHNICAL FIELD

The present invention relates to a method for preparing an intracellular enzyme. More specifically, the invention relates to a method for easily preparing an intracellular enzyme of yeast. The present application claims priority based on Japanese Patent Application No. 2015-110689 filed on May 29, 2015, and the entire contents of the patent application are incorporated herein by reference.

### BACKGROUND ART

Currently, the industrial utilization of enzymes in medical and food fields has become active. The “enzyme” is a generic name for proteinaceous catalysts for promoting chemical reactions of digestion/absorption of substances and the like. Enzymes are substances which exist in organisms and are essential for living, and have been utilized in the production of alcoholic liquors such as beer and wine and fermented foods such as cheese and yogurt in the food field. In recent years, research and development of foods in which specific substances are activated by enzymes and beneficially act on human bodies have been carried out vigorously. Among those foods, milk foods are attracting attention. Milk foods are indispensable in human life, and are nutritious foods abundantly containing proteins as well as carbohydrates, vitamins and the like. The digestive enzyme which is necessary to ingest this milk product is a lactase. Milk foods are absorbed into human bodies by a lactase decomposing the lactose contained in the milk foods into galactose and glucose. However, there are not a few lactose-intolerant persons who congenitally lack lactase and thus cannot ingest milk foods. Hence, there have been carried out research and development of low-lactose products in which lactose has been decomposed in advance by a lactase for lactose-intolerant persons.

Yeast-derived lactases exist in the cytoplasm (i.e., produced as intracellular enzymes), and are not secreted to the outside of cell bodies. Therefore, sonication or the like is used for recovery of the yeast-derived lactases. The cell membrane of yeast, however, is very hard, and cannot be taken out unless glass beads having polishing action or the like are used in combination. In conventional methods, the efficiency of sonication is increased by use of glass beads or the like in combination, and the cell membrane is broken while yeast is cooled to dissolve the lactase in a culture solution. Thereafter, the cell bodies are removed, and the resultant solution is utilized as an enzyme solution. Examples of disadvantages of the sonication method include complicated enzyme extraction steps and associated increase in treatment time, and concerns about reduction in enzyme activity caused by physical impacts. The techniques in which a pulsed electric field is utilized, for example, in the modification and control of microorganisms and cells are cited below (Patent Documents 1 to 3).

### PRIOR ART DOCUMENTS

### Patent Documents

Patent Document 1: Japanese Unexamined Patent Application Publication No. H06-277060

Patent Document 2: Japanese Unexamined Patent Application Publication No. 2012-213353

Patent Document 3: Japanese Unexamined Patent Application Publication No. 2013-236600

### SUMMARY OF THE INVENTION

### Problem to be Solved by the Invention

Yeast-derived enzymes are utilized in various fields. The preparation of such enzymes, however, involves the problem as mentioned above. For further use/utilization of yeast-derived enzymes, it is desired to provide a simpler means for extracting an intracellular enzyme. Thus, an object of the present invention is to prepare an intracellular enzyme of yeast by a simple method.

### Means for Solving Problem

The present inventors made earnest examination in order to solve the above-indicated problem. Specifically, the inventors attempted to apply a specific pulsed electric field to a solution containing a yeast cell to extract the intracellular enzyme of interest (lactase) to the outside of the cell body. As a result, it has been found that the application of the pulsed electric field was effective in the extraction of the enzyme. Also, it has been found that, when the cell body was transferred into phosphate buffered saline as an isotonic solution after the application of the pulsed electric field, the extraction of the enzyme was promoted, leading to the improvement in recovery rate. Further, information useful for efficient enzyme extraction, such as pulsed electric field conditions, was obtained.

The following inventions are based mainly on the above-mentioned findings.

[1] A method for preparing an intracellular enzyme of yeast, comprising the following steps (1) and (2):

(1) applying a pulsed electric field to yeast; and

(2) recovering the enzyme extracted into an extracellular solution.

[2] A method for preparing an intracellular enzyme of yeast, comprising the following steps (1) and (3):

(1) applying a pulsed electric field to yeast; and

(3) transferring the yeast after the step into an isotonic solution, leaving the yeast as it is, and then recovering the enzyme extracted into the isotonic solution.

[3] The preparation method according to [2], wherein the isotonic solution is phosphate buffered saline.

[4] The preparation method according to any one of [1] to [3], wherein the pulse waveform of the pulsed electric field is a damped oscillatory waveform.

[5] The preparation method according to any one of [1] to [4], wherein the electric field strength of the pulsed electric field ranges from 10 kV/cm to 50 kV/cm.

[6] The preparation method according to any one of [1] to [5], wherein the number of times of application of the pulsed electric field is more than one time.

[7] The preparation method according to any one of [1] to [6], wherein the yeast is Kluyveromyces lactis. [8] The preparation method according to any one of [1] to [7], wherein the intracellular enzyme is a lactase.

### Effect of the Invention

The preparation method of the present invention requires steps less than those of conventional methods (a sonication step with the use of glass beads or the like in combination is carried out), and can achieve simplification of the treatment step and shortening of the treatment time. Also, since treatment can be carried out under mild conditions as compared with the sonication, the method can suppress damage to the enzyme of interest, so that the increase in quantity of activity to be recovered can be expected. Further, the treatment is not accompanied with crushing of cell bodies in the present invention, and thus it is also made possible to extract the enzyme of interest while maintaining (keeping alive) the cell bodies.

### BRIEF DESCRIPTION OF THE DRAWINGS

FIG. 1 shows one example of a pulsed electric field generator which can be used in the present invention.

FIG. 2 shows one example of a pulsed voltage waveform to be applied in the present invention.

FIG. 3 shows results of measurement of the enzyme activity (lactase activity). After application of a pulsed electric field to the yeast after culture at an electric field strength of 10 kV/cm, 20 kV/cm or 30 kV/cm, the activity of the enzyme in the culture solution was measured. The enzyme activity without application of a pulsed electric field was used as a control. For comparison, the activity of the enzyme in the culture solution when the yeast after culture was sonicated was also measured.

FIG. 4 shows results of measurement of the enzyme activity (lactase activity). Compared were the activity of the enzyme in the culture solution when a pulsed electric field was applied to the yeast after culture and the enzyme activity (entire lactase activity) when the yeast after culture was ground to extract the enzyme.

FIG. 5 shows results of measurement of the enzyme activity (lactase activity). Measured was the activity of the enzyme released into each solvent by applying a pulsed electric field, then inoculating yeast in water, a medium or physiological saline, and leaving the yeast as it was. The measurement results were evaluated based on the proportion (%) with respect to the entire lactase activity.

FIG. 6 shows results of measurement of the enzyme activity (lactase activity). Measured was the activity of the lactase released into a supernatant when a test sample without application of an electric field was washed, transferred into a mortar, and ground for 30 minutes with addition of 1 g of glass beads thereto, and, thereafter, the cell concentration was adjusted to 1.0×10⁹ CFU/mL.

### DESCRIPTION OF EMBODIMENTS

The present invention relates to a method for preparing an intracellular enzyme of yeast. In one embodiment of the present invention, the following steps (1) and (2) are carried out.

(1) the step of applying a pulsed electric field to yeast

(2) the step of recovering the enzyme extracted into an extracellular solution

In step (1), a pulsed electric field is applied to yeast. Examples of the yeast include Kluyveromyces lactis, K. marxianus, Saccharomyces cerevisiae, Sporobolomyces singularis, Cryptococcus, and Pichia pastoris. The yeast to be used is not particularly limited so long as the yeast produces the enzyme of interest. One example of suitable yeasts is Kluyveromyces lactis. In the present invention, an intracellular enzyme is prepared. In other words, the enzyme of interest in the present invention is an intracellular enzyme. Any intracellular enzyme with industrial usability can be employed as the enzyme of interest. For example, lactase, α-amylase, peptidase or the like is employed as the enzyme of interest. Lactase is referred to also as β-galactosidase from the prefix of lactose. Industrially, it is collected mainly from yeasts such as Kluyveromyces lactis and microorganisms such as Bacillus circulans (spore-forming bacteria) and Aspergillus oryzae (mold), which have been confirmed to be safe. Among the digestive organs of humans, it exists abundantly in the small intestine. When lactose is not decomposed in the intestines due to lack of lactase, fermentation progresses due to enterobacteria, with the result that lactose turns into carbon dioxide gas and fatty acids which stimulate the intestines. This is a cause of disorders.

In step (1), a pulsed electric field is applied to yeast in a state of existing in an appropriate solvent (referred to also as “extracellular solution” herein for comparison with/distinction from the intracellular solution). Typically, a pulsed electric field is applied to yeast in a state of being suspended in a culture solution (for example, yeast during or after culture), yeast in a state of being recovered after culture and suspended in another solvent (for example, a buffer), etc.

Examples of the extracellular solution used in the application of the pulsed electric field include, but not limited to, culture solutions, physiological saline, various buffers, and pure water. For example, the pulsed electric field is applied via an electrode provided in an appropriate container in which a yeast-containing solution (for example, yeast suspension) is housed. Continuous treatment may also be carried out by providing a flow passage having an electrode disposed therein and flowing a yeast-containing solution into the flow passage (circulating the solution, as needed).

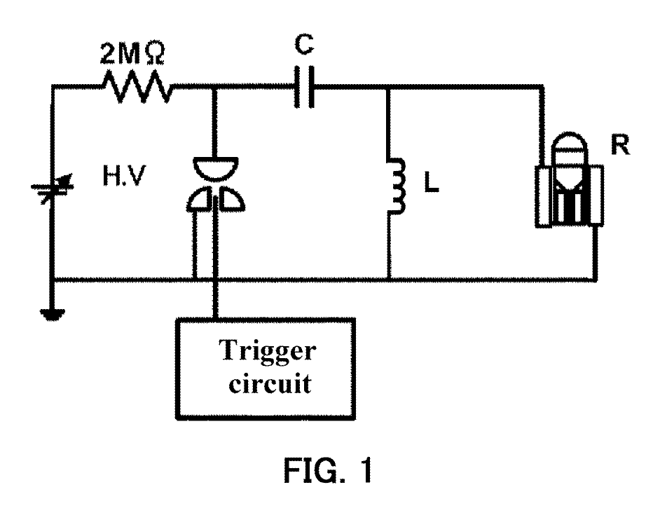

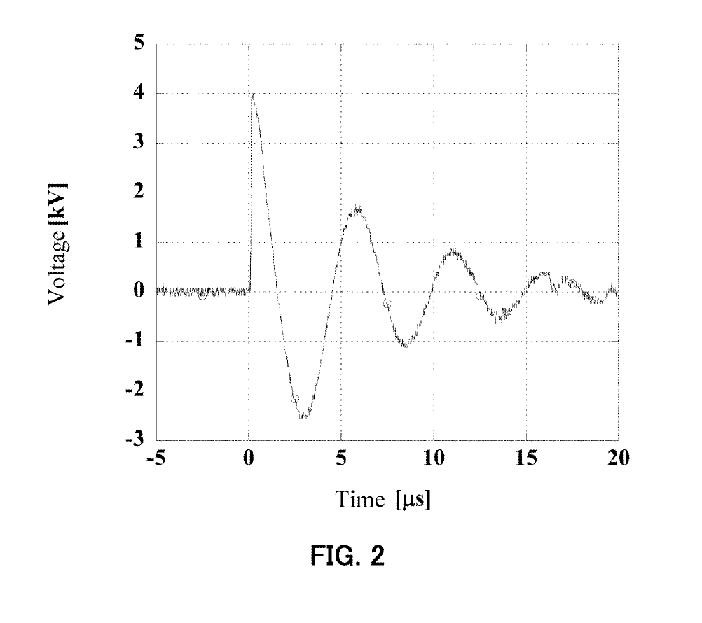

FIG. 1 shows one example of the circuit of a pulsed electric field generator usable in the present invention. FIG. 2 shows one example of the pulse waveform output by this device. This device is composed of a high-voltage power source, a resistance (2 Me), a capacitor C, an inductance L, a trigatron gap switch, and a trigger circuit, and L and C constitute a parallel resonance circuit. The capacitor to be used is C=90 nF.

An operation principle will now be explained. At the beginning, electric charge is charged through a 2-Me resistance into a capacitance C by a high-voltage power source. After charge, a gap switch is used to cause discharge, so that the electric charge charged in C is released into an RLC circuit. The current flowing into the RLC circuit forms a damped oscillatory waveform by resonance between C and L, and is output to R which is a sample solution connected in parallel.

While the damped oscillatory waveform shown in FIG. 2 is output by this pulsed electric field generator, a non-oscillatory damped waveform can also be output by employing a circuit without the inductance L. Such a device can also be used in the present invention.

To minimize influences of heat generated by application of the pulsed electric field, it is effective to install a water cooling device for cooling an electrode part. For example, a water cooling device is installed in such a manner that water flows into an electrode on the ground side by means of a pump, thereby cooling the electrode on the ground side. Further, a cooling fin for heat exchange is mounted on the high-voltage side for easy heat dissipation.

Such a configuration can suppress a rise in temperature of the sample during electric field application.

Upon application of a pulsed electric field to cells, electric charge is accumulated in the cell membrane which works as a capacitor in the electric properties of the cells. Thus, a potential difference is caused between the outside and inside of the cell membranes. When an electric field having an electric field strength E is applied to a cell having a radius a, the potential difference Vm applied to the membrane located in a position forming an angle θ with the electric field direction is expressed according to the following formula. The potential difference is proportional to the diameter of the cell and the electric field strength, and varies depending on the membrane position with respect to the electric field direction.

Vm=1.5a·E·cos θ [Formula 1]

When this potential difference exceeds 1 V, the cell membrane causes dielectric breakdown. The dielectric breakdown of the cell membrane leads to formation of pores in the cells. Such formation of pores in the cells by a pulsed electric field is referred to as electroporation. The potential difference of 1 V generates a very large electric field of 2×10⁶ V/cm in the cell membrane. This pore, if being not so large, is reversible breakdown which is repaired by cells themselves. However, when the energy to be added is increased, for example, by increasing the electric field strength or the pulse width, there occurs irreversible cell membrane breakdown which cannot be repaired by cells themselves any more. Then, the tissue in the cells flows out, leading to necrosis of the cells. Since the potential difference applied to the cell membrane becomes larger as the cells have a larger diameter, the cell membrane is easily broken. For example, yeast has a diameter larger than that of E. coli, and thus the potential difference applied to the cell membrane becomes larger when a pulsed electric field is applied.

The electric field strength of the pulsed electric field is not particularly limited so long as pores which enable the release of an intracellular enzyme can be formed in the cell membrane, but, for example, is 10 kV/cm to 50 kV/cm, preferably 10 kV/cm to 30 kV/cm, more preferably 20 kV/cm to 30 kV/cm. Also, the pulsed electric field is preferably applied more than one time. So, the number of times of application is defined, for example, within the range of 10 shots (times) to 10,000 shots (times), preferably 100 shots (times) to 2,000 shots (times), more preferably 100 shots (times) to 1,500 shots (times). The number of repetitions can be set within the range where the temperature of the solution would not be raised, for example, the range of from 1 pps to 1,000 pps.

The intracellular enzyme of interest is released (extracted) into an extracellular solution by step (1). In the subsequent step (2), the enzyme of interest extracted into the extracellular solution is recovered. In the present invention, the enzyme of interest is released into the extracellular solution (for example, culture solution), and thus the enzyme of interest can be recovered from the extracellular solution without crushing of cell bodies. Accordingly, the enzyme of interest can be recovered remarkably simply and easily as compared with conventional recovery methods accompanied with crushing of cell bodies by sonication (glass beads or the like are used in combination). While the recovery operation in step (2) is not particularly limited, cell bodies are removed by filtration, centrifugation or the like to obtain a solution containing the enzyme of interest. Further, a purification step such as concentration, dilution, salting-out, dialysis, dissolution, adsorption and elution, and drying may be carried out to obtain a high-purity enzyme.

In another embodiment of the present invention, the following step (1) and (3) are carried out.

(1) the step of applying a pulsed electric field to yeast

(3) the step of transferring the yeast after the step into an isotonic solution, leaving the yeast as it is, and then recovering the enzyme extracted into the isotonic solution

Step (1) in this embodiment is the same as that in the above-mentioned embodiment, and thus is not explained. Step (3), which is characteristic of the embodiment, will now be explained. In step (3), yeast is transferred into an isotonic solution after step (1), and left as it is. By this operation, an intracellular enzyme is released into the isotonic solution. Examples of the isotonic solution include phosphate buffered saline, physiological saline and various buffers. While the time for leaving the yeast to stand is not particularly limited, but is defined, for example, within the range of from 1 hour to 3 days, preferably from 5 hours to 2 days. When the time for leaving the yeast to stand is too short, an enough amount of the intracellular enzyme cannot be released. When the time is too long, on the other hand, the enzyme is likely to be deactivated. The yeast is preferably left as it is under low-temperature conditions, for example, conditions of 4° C. to 20° C., preferably 4° C. to 10° C., to prevent the deactivation of the enzyme.

The recovery of the enzyme extracted into the isotonic solution may be carried out through operations similar to those in step (2) in the above-mentioned embodiment.

Hereinafter, Examples (experimental examples) of the present invention will be illustrated, but the present invention would not be limited thereby.

### EXAMPLES

### (Test Sample)

Yeast Kluyveromyces lactis (k. lactis) was used in this experiment. K. lactis is budding yeast which produces an intracellular lactase, and has a size of 3 μm to 4 μm. The yeast was cultured at a temperature of 28° C. By culturing for 48 hours, the cell concentration was adjusted to about 1.0×10⁸ cells/mL. This yeast solution was adjusted so that the cell concentration was about 1.0×10⁹ cells/mL. After addition of physiological saline, the solution was centrifuged (4,500 rpm, 15 min) for washing, and the cell concentration was adjusted to 1.0×10⁹ CFU/mL with a liquid medium, thereby obtaining a sample solution to be used in the following experiment.

### 1. Example 1

### (Application of Pulsed Electric Field)

The sample solution was charged in a 2-mm gap electroporation cuvette, and a pulsed electric field was applied thereto. The application conditions were: electric field strength of 10 kV/cm, 20 kV/cm or 30 kV/cm; number of times of application of 100 shots (shots); and number of repetitions of 1 pps.

### (Measurement)

For comparison, a solution without application of an electric field was employed as a control sample. On the other hand, the sample solution was also compared with a solution obtained by transferring yeast into a mortar after washing, adding 1 g of glass beads thereto, grinding the mixture for 30 minutes to expose all lactases in the yeast, and thereafter adjusting the cell concentration to 1.0×10⁹ CFU/mL with ultrapure water. The lactase activity value of this solution represents the activity of all the lactases contained in the yeast.

The enzyme activity was measured through the following procedures. After application of the pulsed electric field, 100 μL of the enzyme sample was charged in 400 μL of an ONPG solution (phosphate buffer: 10 mL, ONPG: 0.037 g) preliminary warmed at 37° C. for 10 minutes to cause a reaction. After the respective times, the reaction was stopped by addition of 500 μL of an aqueous sodium carbonate solution, and the solution was diluted with ultrapure water. This was employed as a sample solution, and its absorbance was measured. The enzyme activity value is calculated from the absorbance according to the following formula. In the formula, A420 is an absorbance at a wavelength of 420 nm, 4.6 is a molecular extinction coefficient, and n is a dilution rate.

[Formula 2]

Enzyme activity value [U/mL]=(ΔA420×amount of reaction solution×n)/(4.6×reaction time×amount of enzyme sample solution) (1)

The following table indicates values used in Formula (1) in the respective experiments.

TABLE 1

| | | | Amount of |
|---|---|---|---|
| | Amount of | Reaction | enzyme sample |
| | reaction | time | solution |
| | solution [mL] | [min.] | [mL] |
| Cell solution | 4 | 30 | 0.1 |
| Supernatant | 3 | 240 | 1 |
| solution | | | |

For using Formula (1), the decomposition of the substrate by the enzyme is required to be constant with respect to the time. Specifically, this formula can be used only for a time which provides a constant slope of the graph which indicates the results of measurement of the absorbance at a wavelength of 420 nm for each time. In this experiment, the slope was constant until 30 minutes for the yeast sample solution and until 240 minutes for the supernatant solution, and thus the reaction time was defined as 30 minutes for the yeast sample solution and 240 minutes for the supernatant solution.

### (Results)

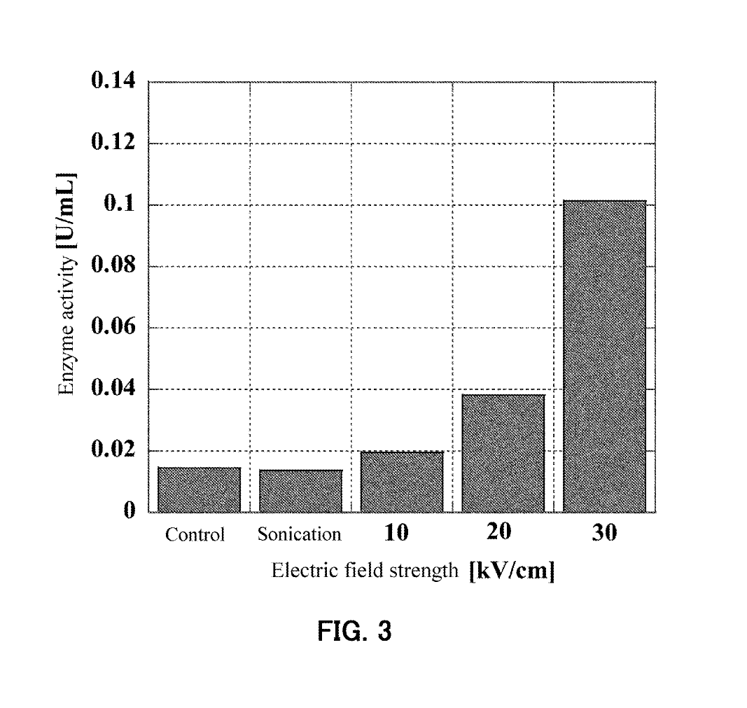

FIG. 3 shows the relation between the electric field strength and the enzyme activity value for the cell solution (containing cell bodies) to which the pulsed electric field was applied. The enzyme activity value increased with the increase in electric field strength, and the solution showed the maximum activity value at an electric field strength of 30 kV/cm. Under all the conditions, the enzyme activity increased as compared with that of the control without application of an electric field.

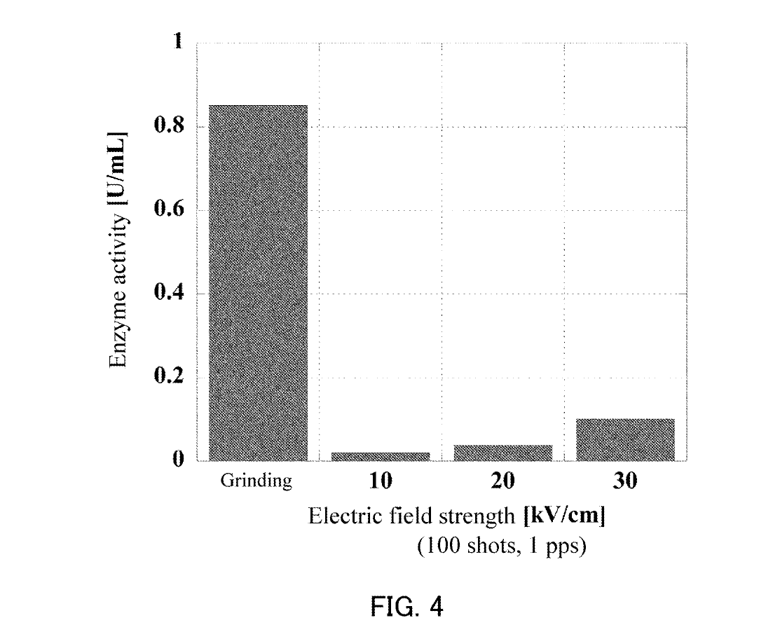

The enzyme activity value when the yeast having a cell concentration of 1.0×10⁹ CFU/mL was ground, i.e., the activity value by all the lactases contained in the yeast was 0.851 U/mL. FIG. 4 shows a comparison between this enzyme activity value and the enzyme activity value of the cell solution after application of the pulsed electric field. The cell bodies to which the pulsed electric field was applied can expose ⅛ of the lactases contained in the yeast under the application condition at this time, i.e., the condition that the number of times of application was 100 shots.

### 2. Example 2

### (Test Sample)

A solution obtained through operations similar to those in Example 1 was used as a sample.

### (Application of Pulsed Electric Field)

The application conditions are: electric field strength: 20 kV/cm, number of times of application: 1,500 shots, and number of repetitions: 1 pps.

### (Measurement)

After pulse application, cell bodies were inoculated into a petri dish containing water, a medium, or phosphate buffered saline, and were left as they were in a refrigerator (4° C.) for 24 hours. After leaving the cell bodies as they were, the solution was centrifugated, and the supernatant was used as an enzyme sample to measure the absorbance in accordance with the method described in Example 1. The results were represented based on the proportion with respect to the enzyme activity value of all lactases when a test sample without application of an electric field was washed and then transferred into a mortar, 1 g of glass beads were added thereto, the mixture was ground for 30 minutes, and then the cell concentration was adjusted to 1.0×10⁹ CFU/mL with ultrapure water.

### (Results)

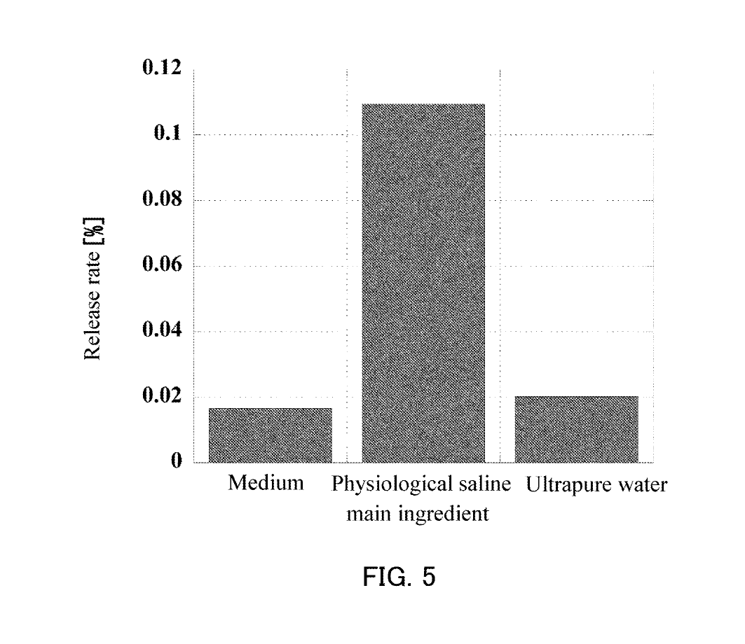

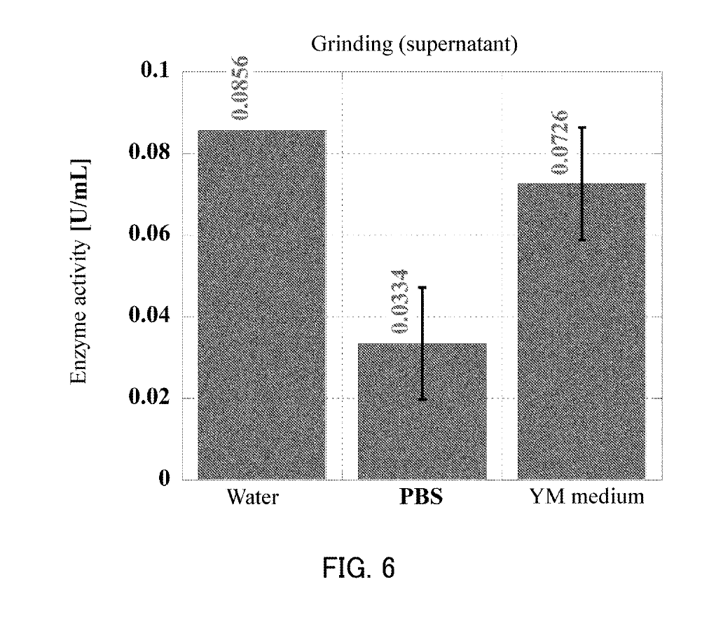

FIG. 5 shows enzyme activity values when the pulsed electric field was applied. The release rate is represented based on the proportion with respect to the enzyme activity value by all the lactases contained in the yeast. In the samples with addition of the pulsed electric field, 0.1% of the enzyme activity value of all the lactases in the yeast was released into the supernatant solution in the sample left as it was in phosphate buffered saline. It is inferred that the improvement in release rate when phosphate buffered saline was used, as compared with the cases where a medium and ultrapure water were used, would be due to easiness to release the enzyme because the osmotic pressure within the yeast and the osmotic pressure of phosphate buffered saline were close to each other. FIG. 6 shows the activity value of the lactase enzyme released into the supernatant when all the lactase enzymes contained in the yeast were exposed by grinding. The enzyme activity value of the lactases released into the supernatant by grinding was 1/10 of the activity value of all the lactase enzymes contained in the yeast (FIG. 4). In brief, the lactase activity of 10% was released into the supernatant when the yeast was ground. From a comparison between this result and the result shown in FIG. 5, it can be understood that the enzyme could be released in an amount corresponding to 1% of the enzyme released into the supernatant when the yeast was ground, by application of the pulsed electric field.

As presented in the above-indicated experimental results, the application of a pulsed electric field was effective as a means for releasing (extracting) a lactase from yeast.

### INDUSTRIAL APPLICABILITY

The present invention makes it possible to extract an intracellular enzyme of yeast by a simple method as compared with conventional methods (a sonication step using glass beads or the like in combination is carried out). The release rate can be improved when, after application of a pulsed voltage, cell bodies are transferred into an isotonic solution (for example, phosphate buffered saline) and left as they are. The application of the present invention to various enzymes can be expected as a means for extracting or preparing an intracellular enzyme produced by yeast.

The present invention is not limited to the above embodiments and Examples. The present invention includes various modifications that can be easily conceived by those skilled in the art without departing from the claims. The entire contents of literatures, patent application publications, and patent publications cited in this description are incorporated herein by reference.

1. A method for preparing an intracellular enzyme of yeast, comprising the following steps (1) and (2):
(1) applying a pulsed electric field to yeast; and
(2) recovering the enzyme extracted into an extracellular solution.

2. A method for preparing an intracellular enzyme of yeast, comprising the following steps (1) and (3):
(1) applying a pulsed electric field to yeast; and
(3) transferring the yeast after the step into an isotonic solution, leaving the yeast as it is, and then recovering the enzyme extracted into the isotonic solution.

3. The preparation method according to claim 2, wherein the isotonic solution is phosphate buffered saline.

4. The preparation method according to claim 1, wherein the pulse waveform of the pulsed electric field is a damped oscillatory waveform.

5. The preparation method according to claim 1, wherein the electric field strength of the pulsed electric field ranges from 10 kv/cm to 50 kv/cm,

6. The preparation method according to claim 1, wherein the number of times of application of the pulsed electric field is more than one time.

7. The preparation method according to claim 1, wherein the yeast is Kluyveromyces lactis.

8. The preparation method according to claim 1, wherein the intracellular enzyme is a lactase.
